(19) United States
(12) Patent Application Publication
(10) Pub. No.: US 2024/0390660 A1
(43) Pub. Date: Nov. 28, 2024

(54) APPARATUS, SYSTEMS AND METHODS FOR TRANSDERMAL DRUG DELIVERY, PERMEABILITY ENHANCEMENT AND AUGMENTATION

(71) Applicant: University of Iowa Research Foundation, Iowa City, IA (US)

(72) Inventors: Nicole Brogden, Coralville, IA (US); Krishna Kumar Patel, Iowa City, IA (US)

(21) Appl. No.: 18/696,636

(22) PCT Filed: Sep. 29, 2022
(86) PCT No.: PCT/US2022/045237

§ 371 Date: Mar. 28, 2024

Related U.S. Application Data
(60) Provisional application No. 63/250,124, filed on Sep. 29, 2021.

(51) Int. Cl. A61M 37/00

(52) U.S. Cl. CPC A61M 37/0015; A61M 2037/0007; A61M 2037/0023; A61M 2037/0046; A61M 2202/0007; A61M 2205/0244; A61M 2205/364; A61M 2210/04

(57) ABSTRACT

The disclosed apparatus, systems and methods relate to a permeation enhancing transdermal patch for the administration of a therapeutic compound transdermally. A transdermal patch for the administration of a therapeutic compound to a subject that has a microneedle layer with microneedles made of naloxone and a binder, an active layer with naloxone and a carrier and an augmentation layer configured to generate local heat on the skin of the subject. The patch can omit the microneedle layer or augmentation layer.

### CROSS-REFERENCE TO RELATED APPLICATIONS

This application claims priority to U.S. Provisional Application No. 63/250,124 filed Sep. 29, 2021, entitled “Apparatus, Systems and Methods For Transdermal Drug Delivery,” which is hereby incorporated by reference in its entirety under 35 U.S.C. § 119 (e).

### GOVERNMENT SUPPORT

This invention was made with government support under R35 GM124551 awarded by the National Institutes of Health. The Government has certain rights in the invention.

### TECHNICAL FIELD

The disclosed technology relates generally to apparatus, systems, and methods for the delivery of a compound, composition or other substance to a subject, person in need thereof and/or patient via a delivery device and associated method. Various implementations relate to the use of the disclosed technology in emergency settings, such as in the field in response to a drug overdose.

In various implementations, the disclosed implementations relate to a transdermal delivery device and method of use that allows for the application of a single-use device to the subject for a rapid and/or sustained transdermal infusion/absorption of a drug and/or therapeutic compounds. For example, certain implementations relate to the application of a delivery patch that comprises one or more layers, an activation system and/or microneedles for the transdermal application of a pharmaceutical such as naloxone. Further implementations are described herein.

### BACKGROUND

For the first time in US history, a person is more likely to die from an opioid overdose than a motor vehicle accident. Opioid overdose is killing people at the level of a #2420358 mass casualty event: the final death toll in Louisiana after Hurricane Katrina was 1,577 and the 2018 death toll from opioid overdose in California alone was 5,348. Increased use of more potent opioids such as fentanyl and its derivatives are causing deaths to continue rising. Despite the urgency and clear clinical need, commercially available preparations of naloxone (the antidote for opioid overdose) are not appropriate to be readily used in all settings, by all individuals (medical personnel or civilians).

Current naloxone (NLX) delivery methods have notable challenges in pre-hospital settings. Most specifically, the need for repeated (“rescue”) doses is becoming increasingly common in order to prevent overdose relapse when a patient has ingested large amounts of opioids. This is especially important when medical care is not yet available.

Due to its physicochemical properties, NLX cannot normally pass through the skin well enough on its own to be clinically effective. Further, the established routes of administration—intravenously (IV), intramuscularly (IM), subcutaneously (SC), or intranasally—can be cumbersome or difficult to achieve in field settings.

NLX also suffers from a very short plasma half-life, presenting risk of overdose reoccurring if the plasma opioid concentration is high or the opioid in the plasma has a longer plasma half-life than NLX. This results in need for multiple NLX doses (“rescue” doses) to prevent hypoxia, and this disadvantage is shared by all routes of delivery. A recent analysis concluded that 33% of intranasal cases and 17% of IM/IV cases require rescue doses. IV, IM, and SC routes suffer from a limitation that they require medical training and use of needles/syringes (increasing risk for needle stick injuries). Intranasal delivery requires specific physical positioning of the victim and cannot be used in the context of intranasal damage, often seen from use of other illicit substances.

Transdermal delivery overcomes all of these limitations and is especially suitable for pre-hospital settings. Patches can be used in all patient populations, including pediatrics, geriatrics, and pregnancy, and no medical training is necessary (nearly everyone is familiar with patches like Band-aids). Sustained delivery would eliminate need for rescue doses while victims and loved ones wait for medical care, and patches are thin and can be easily stored in first-aid kits, purses, or a wallet-making them accessible nearly everywhere.

There is a critical need to develop a NLX dosage form that could be readily used in pre-hospital settings and does not require rescue doses for ongoing effectiveness. The disclosed technology addresses these challenges via a self-heating transdermal patch with embedded microneedles. This has implications beyond the application of NLX, as will be readily apparent from the disclosed implementations and description.

There is further need in the art to provide a flexible transdermal platform for the administration of a therapeutic compound over a prescribed time course, be it rapid onset or prolonged exposure.

### BRIEF SUMMARY

Discussed herein are various devices, systems and methods relating to a transdermal NLX patch with microneedles and a self-heating layer will overcome these challenges.

In Example 1, a transdermal patch comprising one or more permeation enhancement layers.

In Example 2, the patch of Example 1, wherein transdermal patch further comprises an active layer and the one or more permeation enhancement layers comprises one or more of a microneedle layer and an augmentation layer.

In Example 3, the patch of Example 1 or 2, comprising an active layer comprising naloxone and a gel.

In Example 4, a transdermal patch comprising a microneedle layer, an active layer, and an augmentation layer.

In Example 5, the patch of Example 4, wherein the microneedle layer and active layer comprise a drug.

In Example 6, the patch of Example 5, wherein the augmentation layer is configured to generate heat.

In Example 7, the patch of Example 6, wherein the active layer comprises naloxone.

In Example 8, the patch of any of Examples 4-7, wherein the microneedle layer comprises dissolving microneedles comprising naloxone.

In Example 9, a transdermal patch for administration of naloxone to a subject in need thereof, comprising a dissolving microneedle layer, an active layer, and an augmentation layer.

In Example 10, the transdermal patch of Example 9, wherein the dissolving microneedle layer further comprises a binder and a hydrophilic drug.

In Example 11, the transdermal patch of Example 10, wherein the binder is selected from polyvinyl pyrrolidone (PVP), polymethyl vinyl ether/maleic anhydride (PMVE/MA), carboxymethylcellulose (CMC), sugars, carbohydrates, dextrin, dextran, sodium hyaluronate, hyaluronic acid (HA), chitosan, sodium carboxymethylcellulose, hydroxypropyl methylcellulose, sodium alginate, polyvinyl alcohol (PVA), hyaluronic acid, cross-linking methacrylates, (hydroxyethyl) methacrylate, HEMA, poly (lactic-co-glycolic) acid (PLGA), poly(styrene)-block-poly (acrylic acid) (PS-b-PAA), PCL, poly (ethylene glycol) diacrylate (PEGDA), polyglycolide (PGA) and polyether ether ketone (PEEK).

In Example 12, the transdermal patch of any of Examples 9-11, wherein at the dissolving microneedle layer and active layer comprise a therapeutic compound.

In Example 13, the transdermal patch of Example 12, wherein the therapeutic compound is a hydrophilic drug.

In Example 14, the transdermal patch of Example 12 or 13, wherein the therapeutic compound is a therapeutic compound for the treatment for one or more of overdose, migraine, anaphylaxis, epilepsy, allergy, anxiety, asthma and COPD treatments, nausea, vomiting, cancer, diabetes, endocrine disorders, genetic disorders, hormonal disruptions, menopause, arrythmias, bacterial infections, viral infections, skin cancer, ocular disorders, depression, psychiatric disorders, HIV, AIDS, seizure disorders, and blood pressure changes.

In Example 15, the transdermal patch of any of Examples 12-14, wherein the therapeutic compound is at least one of a triptan drug, a muscle relaxant, an antimicrobial, an antibiotic, an antifungal, an anthelminthic, an antihistamine, an allergy medication, an analgesic, a blood pressure medication, a vitamin, an anti-retroviral, a gene therapy, a hormone therapy, an arrythmia therapy, a chemotherapy medication, an anti-depressant, an anti-anxiety medication, an anti-psychotic medication, a medication for attention-deficit/hyperactivity disorder, an anti-diarrheal, a stool softener, a stimulant laxative, an antidote for overdose, a treatment for alcohol use disorder, a treatment for opioid use disorder, a medication for skin disorders, a vaccine and/or a neuromuscular blocking medication.

In Example 16, the transdermal patch of Example 15, wherein the medication for skin disorders is directed to the treatment of psoriasis, atopic dermatitis or eczema.

In Example 17, the transdermal patch of any of Examples 12-15, wherein the therapeutic compound is selected from naloxone, hydrophilic drugs, triptans, muscle relaxants, antimicrobials, antibiotics, antifungals, anthelminthics, antihistamines, allergy medications, analgesics, blood pressure medications, vitamins and neuromuscular blocking medications and further comprising a carrier selected from the group consisting of gel, immediate release gel, extended release gel, solutions, drug matrixes, drug reservoirs, suspensions, creams, nanoparticles, solid dispersions, drug films, adhesive layers, organogels, bigels, emulgels, nanogels, ointments, pastes, lotions, codrugs, prodrugs, cosolvents, surfactants, terpenes and terpenoids, poloxamers, crystals, liquid crystals, cocrystals, supersaturated formulations, polymeric drug carriers, vesicular carriers, microparticles, microcarriers, nanocarriers, micelles, ethosomes, liposomes, dendrimers, lipids, transfersomes, niosomes, emulsions (microemulsions and nanoemulsions), metallic particles, metallic carriers, polysaccharides, nucleic acid carriers, liquid paraffin, eutectic mixtures, alcohols, and water.

In Example 18, the transdermal patch of any of Examples 9-17, wherein the augmentation layer is configured to generate heat.

In Example 19, the transdermal patch of any of Examples 9-18, further comprising a liner, wherein the augmentation layer is configured to generate heat upon removal of the liner.

In Example 20, the transdermal patch of any of Examples 9-19, wherein the active layer comprises a therapeutic compound and a carrier.

In Example 21, a transdermal patch for the administration of naloxone to a subject in need thereof, comprising a microneedle layer, an active layer, and an augmentation layer.

In Example 22, the patch of Example 21, wherein the microneedle layer comprises dissolving microneedles.

In Example 23, the patch of Example 21 or 22, wherein the microneedle layer comprises non-dissolving microneedles.

In Example 24, the patch of any of Examples 21-23, wherein the microneedle layer comprises dissolving microneedles.

In Example 25, the patch of any of Examples 21-24, wherein the microneedle layer comprises dissolving microneedles and non-dissolving microneedles.

In Example 26, the patch of any of Examples 21-25, wherein at least one of the active layer or the microneedle layer comprises a therapeutic compound.

In Example 27, the patch of Example 26, wherein the therapeutic compound is selected from naloxone, almotriptan, eletriptan, frovatriptan, naratriptan, rizatriptan, sumatriptan, zolmatriptan, acetazolamide, clonazepam, diazepam, ethosuximide, fosphenytoin, lamotrigine, levetiracetam, lidocaine, lorazepam, mephobarbital, methsuximide, midazolam, pentobarbital, phenobarbital, phenytoin, piracetam, thiopental, topiramate, valproic acid, antimicrobials, disulfiram, naltrexone, ondansetron, azelastine, fluticasone, beclomethasone, budesonide, cetirizine, ciclesonide, cortisone, cromolyn sodium, desloratadine, dexamethasone, fexofenadine, flunisolide, fluticasone, hydrocortisone, ipratropium, levocetirizine, loratadine, methylprenisolone, mometasone, montelukast, olopatadine, ranitidine, prednisolone, prednisone, triamcinolone, betamethasone, carbinoxamine, cortisone, cyproheptadine, dexamethasone, dexchlorpheniramine, diphenhydramine, epinephrine, hydrocortisone, methylprednisolone, prednisolone, prednisone, acetaminophen, aspirin, codeine, dichloralphenazone, isometheptene, butalbital, caffeine, butorphanol, celecoxib, diclofenac, dihydroergotamine, ergotamine, flurbiprofen, ganaxolone, ketoprofen, ketorolac, rimegepant, ibuprofen, lasmiditan, rofecoxib, naproxen, ubrogepant, spironolactone, acarbose, acetohexamide, albiglutide, alogliptin, alpha lipoic acid, bromocriptine, canagliflozin, chlorpropamide, chromium, colesevelam, dapagliflozin, dulaglutide, empagliflozin, ertugliflozin, exenatide, garlic, allium sativum, glimepiride, glipizide, glyburide, insulin (all types), linagliptin, liraglutide, lixisenatide, metformin, miglitol, muraglitazar, nateglinide, pioglitazone, pramlintide, repaglinide, rosiglitazone, saxagliptin, semaglutide, sitagliptin, tolazamide, tolbutamide, troglitazone, vidagliptin, acebutolol, aliskiren, amlodipine, amiloride, atenolol, azlisartan, benazepril, bendroflumethiazide, betaxolol, bisoprolol, bucindolol, candesartan, captopril, carvedilol, chlorthalidone, chlorothiazide, clevidipine, clonidine, denoldopam, diltiazem, dixazosin, enalapril, enalaprilat, eplerenone, eprosartan, felodipine, fosinopril, furosemide, guanabenz, guanfacine, hydralazine, hydrochlorothiazide, indapamide, irbesartan, isradipine, labetalol, lercanidipine, levamlodipine, lisinopril, losartan, methyldopa, moexipril, mecamylamine, methyclothiazide, metolazone, metoprolol, mibefradil, minoxidil, nadolol, nebivolol, nicardipine, nisoldipine, olmesartan, omapatrilat, penbutolol, perindopril, pindolol, prazosin, propranolol, quinapril, ramipril, reserpine, spirapril, telmisartan, temocapril, terazosin, timolol, torsemide, trandolapril, triamterene, valsartan, verapamil, vitamins, co-enzume Q10, ubiquinone, ethacrynic acid, magnesium salts, abobotulinumtoxin A, baclofen, carisoprodol, cyclobenzaprine, dantrolene, gabapentin, incobotulinumtoxin A, onabotulinumtoxin A, orphenadrine, phenyltoloxamine, salicylamide, tizanidine, ziconotide, alprazolam, amitriptyline, amoxapine, aripiprazole, brexpiprazole, bupropion, buspirone, butabarbital, chlordiazepoxide, ditalopram, clorazepate, dapoxetine, desipramine, desvenlafaxine, diazepam, doxepin, droperidole, duloxetine, escitalopram, esketamine, fluoxetine, flurazepam, fluvoxamine, kava kava, ketamine, gepirone, hydroxyzine, hypericum perforatum, imipramine, isocarboxazid, levomefolate, levomilnacipran, lithium, lorazepam, maprotiline, meprobamate, methylphenidate, midazolam, milnacipran, mirtazapine, modefinil, nadolol, nefazodone, netamiftide, nortriptyline, olanzapine, oxazepam, oxymorphone, pagoclone, paroxetine, pentobarbital, perphenazine, phenelzine, piper methysticum, prazepam, prochlorperazine, propranolol, protriptyline, quetiapine, reboxetine, S-adenosyl-L-methionine, selegiline, sertindole, sertraline, St. John's Wart, tranylcypromine, trazodone, trimipramine, tryptophan, 5-hydroxytryptophan, valerian, venlafaxine, vilazodone, vortioxetine, chlorpromazine, dimenhydrinate, droperidol, hydroxyzine, meclizine, metoclopramide, methylnaltrexone, naltrexone, perphenazine, phosphorated carbohydrate solution, prochlorperazine, promethazine, ramosetron, scopolamine, thiethylperazine and trimethobenzamide.

In Example 28, the patch of Example 26 or 27, wherein augmentation layer comprises one or more of iron, perlite, fine saw dust, fine coir, sodium acetate, graphene oxide and/or nanostructures.

In Example 29, a method of transdermal administration of a therapeutic compound to a subject in need thereof, comprising applying a transdermal patch to skin of the subject, the transdermal patch comprising at least one of a microneedle layer, an active layer, and an augmentation layer, and removing a liner, such that subject skin permeability is enhanced.

In Example 30, the method of Example 29, wherein administration time is less than about 30 minutes.

In Example 31, the method of Example 29, wherein administration time is less than about 15 minutes.

In Example 32, the method of Example 29, wherein administration time is less than about 10 minutes.

In Example 33, the method of Example 29, wherein administration time is less than about 5 minutes.

In Example 34, the method of Example 29, wherein administration time is less than about 1 minute.

In Example 35, the method of Example 29, wherein administration time is more than about 30 minutes.

In Example 36, the method of Example 29, wherein administration time is more than about 60 minutes.

In Example 37, the method of Example 29, wherein administration time is more than about 90 minutes.

In Example 38, a transdermal patch for the administration of naloxone to a subject, comprising a microneedle layer comprising microneedles comprising naloxone and a binder, an active layer comprising naloxone and a carrier, and an augmentation layer configured to generate local heat on skin of the subject.

In Example 39, a transdermal patch for the administration of naloxone to a subject, comprising a microneedle layer comprising microneedles comprising naloxone and a binder, and an augmentation layer configured to generate local heat on skin of the subject.

In Example 40, the transdermal patch of Example 39, further comprising an active layer.

In Example 41, the transdermal patch of Example 40, wherein the active layer comprises naloxone and a carrier.

In Example 42, a transdermal patch for the administration of a therapeutic compound to a subject, comprising a microneedle layer comprising microneedles comprising the therapeutic compound, and an augmentation layer configured to generate local heat on skin of the subject.

In Example 43, a transdermal patch comprising a microneedle layer comprising dissolving microneedles comprising a therapeutic compound.

In Example 44, the transdermal patch of Example 43, wherein the microneedle layer further comprises at least one of non-dissolving microneedles, and/or dissolving microneedles.

In Example 45, the transdermal patch of Example 43 or 44, further comprising an augmentation layer.

In Example 46, the transdermal patch of any of Examples 43-45, further comprising an augmentation layer.

In Example 47, the transdermal patch of any of Examples 43-45, further comprising an active layer.

Various implementations of the disclosed patch include a plurality of layers: dissolving microneedles, a trans-dermal or topical NLX gel, and a heating layer. Each layer fulfills a specific function, with the intent of producing a combined additive or synergistic effect. Dissolving NLX microneedles will immediately start to deliver NLX as they dissolve, while creating micropores to allow transdermal or topical NLX delivery from the gel. The gel will deliver NLX through the micropores for a minimum of 2 hrs. after patch application. The heating layer will rapidly generate heat upon air exposure via, for example, a chemical reaction such as an exothermic reaction, increasing rate and extent of NLX absorption.

This patch design will reduce or eliminate lag time in delivery, and ensure sustained delivery that eliminates the need to re-dose. Microneedles, such as dissolving microneedles, will begin immediate NLX delivery as they dissolve in the skin in ≤5 min (an achievable benchmark with dissolving microneedles). When exposed to air, the heating augmentation layer will increase local temperature of the skin 2 and the gel or carrier, and enhance the rate and extent of NLX delivery from a gel or carrier that will absorb through micropores created by the microneedles. The preliminary in vitro data demonstrate that microneedle pretreatment combined with increased skin temperature permits rapid NLX delivery such that the administration time is less than about 30 minutes or less, while also increasing NLX delivery 3-4× (vs controls) by 1 hour. These combined data support the feasibility of the approach. The goal is to decrease pre-hospital opioid overdose deaths through development of a novel transdermal NLX dosage form. The objective of the current work is to optimize patch parameters to deliver a NLX dose sufficient to mitigate opioid overdose with minimal to no lag time, while rapidly producing heat at the skin surface without damaging the tissue.

The central hypothesis is that the use of the patch for NLX or other therapeutic compounds such that the allow the compound to be rapidly and continuously absorbed through epidermal micropores due to the described permeation enhancements, and a positive correlation will exist between elevated skin temperature and the rate and extent of NLX permeation.

In certain implementations, dissolving microneedles will permit detectable NLX absorption in ≤5 min (vs intact skin). Locally generated heat is able to increase the rate and extent of NLX absorption through the epidermal micropores, achieving maximum concentration within about 15-20 minutes or less. These implementations enhance permeability, decrease administration time, and align with the pharmacokinetics of IM and intranasal delivery of, for example, NLX. Further implementations achieve maximum concentrations in more or less time, such as 5 minutes or less or after an hour or more, as would be readily understood for various implementations featuring other therapeutic compounds. Additionally, it is appreciated that in alternative implementations featuring NLX, time to maximum concentration can be less than about 15 minutes, down to one minute or even less, and in further implementations the range of time to maximum can exceed about 20 minutes, up to an hour or more.

In various implementations, the combined patch layers (dissolving microneedles, NLX gel, heating layer) will synergistically increase NLX peak plasma concentration (Cₘₐₓ) and—where applicable—decrease time to achieve maximum concentration (Tₘₐₓ) such that the administration time via the transdermal patch is comparable to that observed via IV, IM or intranasal administration in some circumstances. It is appreciated that in certain situations the release of the therapeutic compound is continuous, so any reference to Tₘₐₓ is understood to be illustrative but not limiting and is completely optional depending on the specific patch configuration.

The disclosed implementations are focused on a completely unexplored approach to the delivery (transdermal) and formulation (self-heating microneedle patch) of an opioid overdose treatment. This will allow a highly effective antidote to be used in challenging pre-hospital situations where current products have notable disadvantages.

Transdermal NLX delivery will provide sustained antidote delivery, eliminating need for rescue doses and directly addressing the concerns of overdose quickly reoccurring (also known as re-narcotization).

Microneedles and heat have not been previously studied in combination for their effects on rapid NLX delivery for opioid overdose. Successful development of such a patch would have broader implications because transdermal absorption of therapeutic compounds that are not typically applied transdermally because of permeability and other reasons—such as the hydrophilic NLX and others—could have a role in treatment of other diseases. Further therapeutic compounds can of course be used.

Transdermal delivery is not conventionally used for acute or life-threatening situations because of the slow lag time often required for skin absorption to therapeutic plasma levels. However, successful development of the proposed patch with absorption times of ≤5 min via the use of, for example, dissolving microneedles and/or an augmentation layer would expand the use of transdermal dosage forms to acute medical needs and provide a highly useful in-field solution. It is understood that in alternate implementations, the desired effect may in fact be delayed onset, and the augmentation layers can be configured to heat more slowly for the administration of the therapeutic compound in the active layer increases more slowly over time.

While multiple embodiments are disclosed, still other embodiments of the disclosure will become apparent to those skilled in the art from the following detailed description, which shows and describes illustrative embodiments of the disclosed apparatus, systems, and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1A is a perspective view of a patch, according to an exemplary embodiment.

FIG. 1B is a side, cutaway view of a patch showing the dissolving microneedle layer, according to an exemplary embodiment.

FIG. 1C is a side, cutaway view of a patch showing the active layer comprising naloxone gel, according to an exemplary embodiment.

FIG. 1D is a side, cutaway view of a patch showing the augmentation layer according to an exemplary embodiment.

FIG. 2A is a side view of a patch comprising a liner, according to an exemplary embodiment.

FIG. 2B is a side view of the patch, according to FIG. 2A, wherein the liner is being removed.

FIG. 2C is a side view of the patch, according to FIG. 2A, wherein the active layer is being introduced into the skin of a subject and the augmentation layer has been exposed to air.

FIG. 2D is a side view of the patch, according to FIG. 2A, wherein the active introduction of the therapeutic compound into the skin of a subject is being enhanced by the heated augmentation layer.

FIG. 3A is a digital image of the dissolving microneedle layer before insertion into porcine skin.

FIG. 3B is a digital image of the resulting micropores in the skin following following insertion of the microneedles shown in FIG. 3A.

FIG. 4 is a graph showing mean, non-cumulative naloxone permeation into a receiver solution.

FIG. 5A is a graph showing the skin temperature change in two representative heating layer studies in response to the exposure to an in vitro heating pod.

FIG. 5B is a graph showing the skin surface temperature measured during in vitro studies using heating pod composition described in Table 3A (this heating pod was also used in vivo).

FIG. 6A depicts a dissolving microneedle (dMN) array containing 100 needles.

FIG. 6A depicts a NLX-HCL gel, shown as transparent gel loaded into small fabricated patch.

FIG. 6C depicts the assembled patch, wherein the heating pod is the augmentation layer applied atop of the elements of FIGS. 6A-6B to form the fully assembled heating patch, shown when applied to the dorsal surface of a guinea pig. For the 2 dMN studies, 2 of the arrays shown in FIG. 6A were applied side-by-side to insert a total of 200 needles.

FIG. 7 depicts in vivo skin surface temperature in guinea pigs (n=5) when a heating pod was applied during pharmacokinetics studies. Data presented as mean±SD.

FIG. 8 depicts a scatterplot showing that the enhancement ratio of plasma NLX-HCL concentrations in vivo was higher at all timepoints when 2 vs 1 dMNs were used.

FIG. 9 depicts graphical representations of NLX-HCL in plasma of guinea pigs during in vivo studies. Concentrations are normalized by NLX-HCL dose per kg of animal body weight. Data presented as mean±SD.

### DETAILED DESCRIPTION

The various embodiments disclosed or contemplated herein relate to such a transdermal delivery device and related systems and methods of use. In certain of the described implementations, the device and use thereof relate to the administration of the drug naloxone, but those of skill in the art will readily appreciate that the device and methods of use can be used with any of a broad number array of pharmaceuticals.

Certain non-limiting examples include treatments for migraine (“triptan” drugs), anti-epileptic drugs, antimicrobials (antibiotics, antifungals, anthelminthics), antihistamines, allergy medications, treatments of anaphylaxis, analgesic/pain medications, blood pressure medications, vitamins, neuromuscular blocking drugs, muscle relaxants, anti-anxiety medications, asthma and COPD treatments, and nausea/vomiting. It will be appreciated by those of skill in the art that there are many applications for a rapid, transdermal delivery patch and method that can be deployed rapidly in field environments, and that many classes of therapeutic compounds would benefit from the enhanced permeation enabled by the disclosed devices, systems and methods.

Certain non-limiting examples of therapeutic compounds also can include a muscle relaxant, an antimicrobial, an antibiotic, an antifungal, an anthelminthic, an antihistamine, an allergy medication, an analgesic, a blood pressure medication, a vitamin, an anti-retroviral, a gene therapy, a hormone therapy, an arrythmia therapy, a chemotherapy medication, an anti-depressant, an anti-anxiety medication, an anti-psychotic medication, a medication for attention-deficit/hyperactivity disorder, an anti-diarrheal, a stool softener, a stimulant laxative, an antidote for overdose, a treatment for alcohol use disorder, a treatment for opioid use disorder, a medication for skin disorders, a vaccine and/or a neuromuscular blocking medication.

It is readily appreciated by those of skill in the art that there are many situations in which the self-administration of a therapeutic compound is more easily achieved via the application of a transdermal patch, including that in certain social or other public settings the self-administration orally may be undesirable, inconvenient or impossible, and that rapid treatment of certain conditions, such as by way of example panic attacks and other mental health disorders may be best achieved via a transdermal application.

Returning to the examples explored in detail herein, more than 130 people in the United States die every day from an opioid overdose, and in 2017 the life expectancy in the United States declined because of opioid use. This crisis continues to grow in magnitude and the epidemic has reached an unprecedented, devastating level. In 2015 the number of drug overdose deaths exceeded AIDS deaths at the peak in 1995 and increases in overdose deaths since then have been greatest in situations involving synthetic opioids such as fentanyl. Naloxone (NLX, often known by the brand name Narcan®) is the most effective reversal agent for opioid overdose. As an opioid receptor antagonist, NLX displaces opioid molecules from their receptors, reversing the respiratory depression and subsequent lack of oxygen to the brain that ultimately causes opioid overdose deaths. Despite the life-saving benefits of NLX, all currently approved delivery methods have significant shortcomings.

In the event of actual or suspected opioid overdose, non-oral delivery is the only option for NLX administration but there are significant challenges with this in pre-hospital settings. Intranasal dosing requires repositioning of the patient to achieve the correct angle of administration, and it cannot be used when contraindications are present (nasal hemorrhage or intranasal damage from use of other illicit substances).

The intranasal form is also not uniformly effective. Parenteral delivery (IV, IM, and SC) requires medical training and use of needles. The majority of states do not allow emergency medical services (EMS) technicians with only basic life support training to administer parenteral drugs, including NLX. If an EMS provider with advanced life support training is not available to administer parenteral NLX and intranasal is not available, the patient is at increased risk of death.

NLX, among other drugs, does not passively cross the hydrophobic lipids of the outer skin layer (stratum corneum) to achieve clinically meaningful concentrations, and it has not been successfully developed into a commercial transdermal formulation.

Accordingly, there is a need in the art for an alternate route of administration for pharmaceuticals like NLX, such as a transdermal administration device and method like that described herein. While it is appreciated that certain of the disclosed implementations relate to the administration of NLX, many further pharmaceuticals can also be utilized in alternate implementations and uses cases.

The disclosed implementations utilizing NLX will decrease pre-hospital opioid overdose deaths through use of a novel transdermal NLX dosage form. Through the use of the path, NLX will rapidly and continuously absorb through epidermal micropores, and induce a positive correlation between elevated skin temperature and the rate and extent of NLX permeation. This technology has broad implications and implementations as a delivery platform, including for use with other pharmaceuticals, compounds and/or compositions that are desirable for transdermal or topical application.

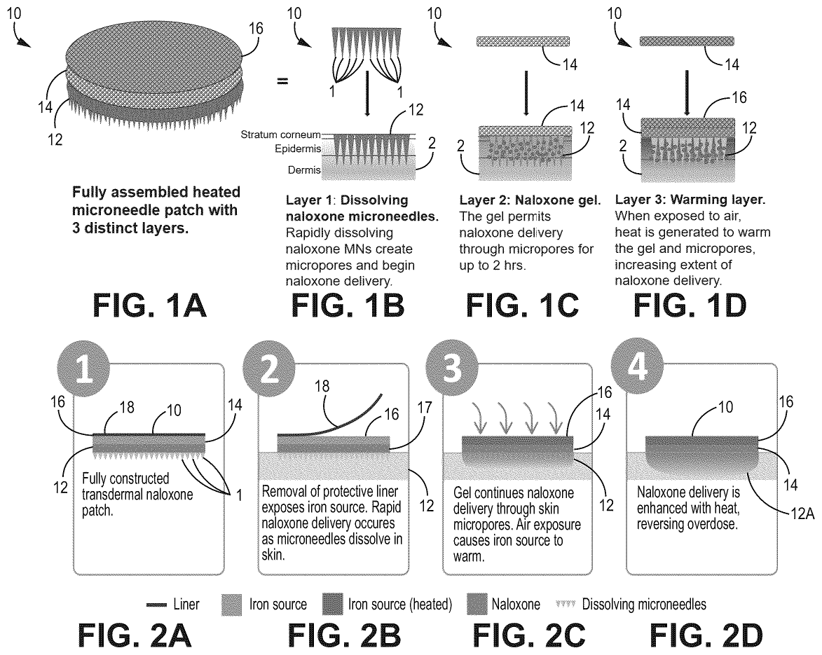

Turning to the drawings in greater detail, FIGS. 1A-1D depict the transdermal patch 10 according to one implementation. In these implementations, the patch 10 comprises a plurality of layers 12, 14, 16. In the implementation of FIGS. 1A-1D, the patch has an optional microneedle layer 12 that, in certain implementations, is a dissolving microneedle layer 12 that, in certain implementations, contains therapeutic compound.

Further, implementations may include an optional an active layer 14 that, in certain implementations, contains therapeutic compound and any inert compounds that may be appropriate for the stability and efficacy of the delivery of the therapeutic compound, as would be readily understood.

Implementations may also comprise an optional augmentation layer 16. Alternate implementations may omit one or more of these layers and/or add additional layers, as will be evident to those of skill in the art from the present disclosure.

In various implementations, the transdermal patch 10 utilizes one or more physical or chemical permeation enhancements, certain non-limiting examples being dissolving microneedles (such as those provided in the dissolving microneedle layer 12 in the implementation of FIGS. 1A-1D) and/or locally generated heat generated by the augmentation layer 16 shown in FIGS. 1A-1D.

In these implementations, the dissolving microneedle layer 12 and active layer 14 can both comprise one or more therapeutic compounds such as a pharmaceutical for administration to the skin 2, such as NLX, or any of the other therapeutic compounds described herein. In certain implementations, dissolving microneedle layer 12 and/or active layer 14 can be omitted, for example in applications where sufficient quantities of therapeutic or pharmaceutical are contained in the dissolving microneedle layer 12 or active layer 14, respectively. Further, in certain implementations, the augmentation layer 16 can be omitted, when the application of heat or other augmentation is not necessary or desirable. Further implementations will be evident from the description here.

According to certain implementations of the patch 10, the therapeutic compound can be any compound directed to the treatment of a disease or diagnosis or need of a subject, patient, victim or other appropriate individual, for example a subject suffering from an overdose or a subject diagnosed with diabetes or anxiety. In various implementations, administration of the therapeutic compound transdermally or topically is enhanced via the permeation enhancements described herein, such as the microneedle layer and/or augmentation layer.

Certain non-limiting examples of therapeutic compounds include the following:
Treatments for migraine, such as almotriptan, eletriptan, frovatriptan, naratriptan, rizatriptan, sumatriptan, zolmatriptan and the like.

Anti-epileptic drugs, such as acetazolamide, clonazepam, diazepam, ethosuximide, fosphenytoin, lamotrigine, levetiracetam, lidocaine, lorazepam, mephobarbital, methsuximide, midazolam, pentobarbital, phenobarbital, phenytoin, piracetam, thiopental, topiramate, valproic acid, divalproex sodium and the like.

Antimicrobials known and understood in the art, such as a substance capable of destroying or inhibiting the growth of microbes, prevents the development of microbes, and/or inhibits the pathogenic action of microbes as well as viruses, fungi, and bacteria.

Alcohol dependence treatments, such as acamprosate, disulfiram, naltrexone, ondansetron, topiramate and the like.

Antihistamines and allergy medications, such as azelastine, fluticasone, beclomethasone, budesonide, cetirizine, ciclesonide, cortisone, cromolyn sodium, desloratadine, dexamethasone, fexofenadine, flunisolide, fluticasone, hydrocortisone, ipratropium, levocetirizine, loratadine, methylprenisolone, mometasone, montelukast, olopatadine, ranitidine, prednisolone, prednisone, triamcinolone and the like.

Treatments of anaphylaxis, such as betamethasone, carbinoxamine, cortisone, cyproheptadine, dexamethasone, dexchlorpheniramine, diphenhydramine, epinephrine, hydrocortisone, methylprednisolone, prednisolone, prednisone, promethazine and the like.

Analgesic/pain medications, such as acetaminophen, aspirin, codeine, dichloralphenazone, isometheptene, butalbital, caffeine, butorphanol, celecoxib, diclofenac, dihydroergotamine, ergotamine, flurbiprofen, ganaxolone, ketoprofen, ketorolac, rimegepant, ibuprofen, lasmiditan, rofecoxib, naproxen, ubrogepant and the like.

Hyperaldosteronism treatments, such as spironolactone, triamterene and the like.

Diabetes therapeutics, such as acarbose, acetohexamide, albiglutide, alogliptin, alpha lipoic acid, bromocriptine, canagliflozin, chlorpropamide, chromium, colesevelam, dapagliflozin, dulaglutide, empagliflozin, ertugliflozin, exenatide, garlic, allium sativum, glimepiride, glipizide, glyburide, insulin (all types), linagliptin, liraglutide, lixisenatide, metformin, miglitol, muraglitazar, nateglinide, pioglitazone, pramlintide, repaglinide, rosiglitazone, saxagliptin, semaglutide, sitagliptin, tolazamide, tolbutamide, troglitazone, vidagliptin and the like.

Blood pressure medications, such as acebutolol, aliskiren, amlodipine, amiloride, atenolol, azlisartan, benazepril, bendroflumethiazide, betaxolol, bisoprolol, bucindolol, candesartan, captopril, carvedilol, chlorthalidone, chlorothiazide, clevidipine, clonidine, denoldopam, diltiazem, dixazosin, enalapril, enalaprilat, eplerenone, eprosartan, felodipine, fosinopril, furosemide, guanabenz, guanfacine, hydralazine, hydrochlorothiazide, indapamide, irbesartan, isradipine, labetalol, lercanidipine, levamlodipine, lisinopril, losartan, methyldopa, moexipril, mecamylamine, methyclothiazide, metolazone, metoprolol, mibefradil, minoxidil, nadolol, nebivolol, nicardipine, nisoldipine, olmesartan, omapatrilat, penbutolol, perindopril, pindolol, prazosin, propranolol, quinapril, ramipril, reserpine, spirapril, telmisartan, temocapril, terazosin, timolol, torsemide, trandolapril, triamterene, valsartan, verapamil and the like.

Vitamins, such as co-enzyme Q10, ubiquinone, ethacrynic acid, magnesium salts and other vitamins understood in the art.

Neuromuscular blocking drugs and muscle relaxants, such as abobotulinumtoxin A, baclofen, carisoprodol, cyclobenzaprine, dantrolene, gabapentin, incobotulinumtoxin A, onabotulinumtoxin A orphenadrine, phenyltoloxamine, salicylamide, tizanidine, ziconotide and the like.

Antianxiety and antidepression medications, such as alprazolam, amitriptyline, amoxapine, aripiprazole, brexpiprazole, bupropion, buspirone, butabarbital, chlordiazepoxide, ditalopram, clorazepate, dapoxetine, desipramine, desvenlafaxine, diazepam, doxepin, droperidole, duloxetine, escitalopram, esketamine, fluoxetine, flurazepam, fluvoxamine, kava kava, ketamine, gepirone, hydroxyzine, hypericum perforatum, imipramine, isocarboxazid, levomefolate, levomilnacipran, lithium, lorazepam, maprotiline, meprobamate, methylphenidate, midazolam, milnacipran, mirtazapine, modefinil, nadolol, nefazodone, netamiftide, nortriptyline, olanzapine, oxazepam, oxymorphone, pagoclone, paroxetine, pentobarbital, perphenazine, phenelzine, piper methysticum, prazepam, prochlorperazine, propranolol, protriptyline, quetiapine, reboxetine, S-adenosyl-L-methionine, selegiline, sertindole, sertraline, St. John's Wart, tranylcypromine, trazodone, trimipramine, tryptophan, 5-hydroxytryptophan, valerian, venlafaxine, vilazodone, vortioxetine and the like.

Nausea/vomiting treatments, such as chlorpromazine, dimenhydrinate, droperidol, hydroxyzine, meclizine, metoclopramide, methylnaltrexone, naltrexone, perphenazine, phosphorated carbohydrate solution, prochlorperazine, promethazine, ramosetron, scopolamine, thiethylperazine, trimethobenzamide and the like.

It is appreciated that many additional therapeutic compounds are contemplated, and that the above examples are in no way comprehensive or intended to be limiting. It is appreciated that the transdermal or topical administration of many classes and types of therapeutics can be enhanced or improved via the permeation enhancements of the various implementations of the patch 10 described herein, for example due to chemical characteristics and functionality.

Continuing with the implementations of FIGS. 1A-1D, the dissolving microneedle layer 12 according to these implementations comprises a plurality of microneedles 1. Here, these microneedles 1 are micro- or millimeter-scale projections configured to painlessly pierce the outer layer of the skin (the skin being shown generally at 2 in the drawings) to allow transdermal delivery of skin impermeable drugs or therapeutic compounds, such as NLX and others, through the skin 2. Dissolving microneedles 1 according to certain implementations deliver a pharmaceutical or other active ingredient or compound such as an encapsulated compound in an immediate and/or sustained release fashion following application to the skin 2.

In various implementations, the dissolving microneedle layer 12/plurality of microneedles 1 comprises one or more therapeutic compounds along with one or more vehicles for dispersing or suspending the therapeutic compound, such as a polymer or other inactive ingredient or binder such as polyvinylpyrrolidone (PVP) or polymethyl vinyl ether/maleic anhydride (PMVE/MA). Further implementations utilize other suitable examples including but not limited to sugars, carbohydrates, dextrin, dextran, sodium hyaluronate, hyaluronic acid (HA), chitosan, sodium carboxymethyl cellulose, carboxymethyl cellulose (CMC), hydroxypropyl methylcellulose, sodium alginate, polyvinyl alcohol (PVA), hyaluronic acid, cross-linking methacrylates, (hydroxyethyl) methacrylate, HEMA; poly (lactic-co-glycolic) acid (PLGA), poly(styrene)-block-poly (acrylic acid) (PS-b-PAA), PCL, poly (ethylene glycol) diacrylate (PEGDA), polyglycolide (PGA), polyether ether ketone (PEEK). Further vehicles would be readily appreciated by those of skill in the art.

While dissolving microneedle layers 12 are described frequently herein, it is appreciated that alternate implementations comprise an optional microneedle layer 12 that is non-dissolving, or a microneedle layer that comprises a combination of dissolving and non-dissolving microneedles. Certain non-limiting examples of non-dissolving microneedles include solid microneedles (e.g., silicon, metal, or plastic), coated microneedles, polymeric microneedles, swellable microneedles, hydrogel-forming microneedles, hollow microneedles, detachable microneedles, bio-inspired microneedles, particle-loaded microneedles, multi-layered microneedles, and the like. It is further appreciated that certain implementations comprise more than one kind of microneedle, including some dissolving and some non-dissolving microneedles, and that the ratios of these dissolving and non-dissolving microneedles may be varied by implementation and in certain aspects can further enhance permeability.

It is also understood that the drug delivery profile of the patch 10 according to certain implementations can be intricately and precisely tailored based on the degradable polymers and ratios selected by the skilled artisan, depending on the specific application. By bypassing the stratum corneum—the outermost, hydrophobic skin layer—such microneedles 1 can painlessly deliver a pharmaceutical or other compound or composition directly to the epidermis or dermis, and reduce or eliminate absorption delays. As a skin-impermeable pharmaceutical with a need for rapid delivery, NLX benefits from dissolving microneedles applied to the skin. It is further appreciated that other pharmaceuticals are of course contemplated. It is readily appreciated that in alternate implementations, the drug delivery profile can also be tailored for extended release, and no statements herein related to rapidity should be taken to be limiting.

It is also understood that pharmaceuticals and therapeutic compounds such as NLX and others mentioned must be delivered directly to the circulatory system for efficacy, which is facilitated by the configuration of the microneedles 1 to penetrate the dermis. The dermis has a rich capillary network that empties directly into the systemic blood supply, and drug absorbed through the skin rapidly enters the circulatory system. Such skin absorption provides consistent, near zero-order delivery over time, which eliminates a need for rescue doses. In various implementations, the more controlled delivery may also be beneficial so a patient does not lose all pain control that that they were taking the opioid for, and it could prevent rapid onset of opioid withdrawal symptoms.

In alternate implementations, the patch 10 is not necessarily transdermal, and can be configured for topical or local use, that is, where there is no need or desire for delivery into the circulatory system. Examples where this would be desirable include localized skin cancer lesions, on fingernails, on the scalp, for pain relief and the like, where systemic absorption is not necessary, as would be readily appreciated. It is appreciated that the skilled artisan would appreciate the preferred delivery onset and duration, and that the various implementations disclosed herein can be configured for the desired release/administration profile.

Continuing with the implementations shown in FIGS. 1A-1D and 2A-2D, a second, active layer 14 is disposed above the microneedle layer 12, opposite the skin 2. In various implementations, the active layer 14 also comprises a pharmaceutical or drug or other active/therapeutic compound, such as NLX, which can optionally be in a carrier such as a gel. That is, and as would be readily understood, in various implementations the microneedle layer 12 and active layer 14 both comprise the desired therapeutic compound for delivery.

In various implementations, the gel or other carrier allow continued NLX delivery after the dissolving microneedles have dissolved/released their drug. For example, NLX in gel will absorb through the micropores that the dissolving microneedles 1 made in the skin 2. Certain non-limiting examples of such a carrier include immediate release gels, extended-release gels, solutions, drug matrixes, drug reservoirs, suspensions, creams, nanoparticles, solid dispersions, drug films, drugs in an adhesive layer, and the like. Additional non-limiting examples for carriers may include organogels, bigels, emulgels, nanogels, ointments, pastes, lotions, codrugs, prodrugs, cosolvents, surfactants, terpenes and terpenoids, poloxamers, crystals, liquid crystals, cocrystals, supersaturated formulations, polymeric drug carriers, vesicular carriers, microparticles, microcarriers, nanocarriers, micelles, ethosomes, liposomes, dendrimers, lipids, transfersomes, niosomes, emulsions (microemulsions and nanoemulsions), polysaccharides, nucleic acid carriers, liquid paraffin, eutectic mixtures, alcohols and water. It is understood that any formulation that would allow the drug to continuously be delivered after the dissolving microneedles can be utilized.

In certain implementations, the device 10 and associated methods can include an excipient or other compound for initiation/augmentation of the administration in the third, heating augmentation layer 16. In various implementations, the augmentation layer 16 is configured to generate local heat at the skin 2 so as to augment the administration of the therapeutic compound to the subject. In various implementations, the augmentation can comprise causing the dissolution of the therapeutic compound and/or increasing the local skin permeability via the application of local heat. Certain implementations also cause the initiation of a chemical reaction, such as an exothermic chemical reaction. In certain implementations, the augmentation layer 16 comprises one or more excipients. In various implementations, the augmentation layer 16 comprises iron, as described herein.

In alternate implementations, the include augmentation layer can include perlite, fine saw dust, fine coir, sodium acetate, graphene oxide, nano-structures, and iontophoresis.

Various implementations of the augmentation layer comprise other excipients.

Certain implementations are configured for various time-sensitive or dependent administration approaches. That is, in certain implementations, rapid administration is desirable, while in other implementations, a time-release or time-delayed or sustained administration is desirable, as would be readily appreciated by those of skill in the art.

In various implementations, the augmentation layer 16 comprises one or more excipients that can be configured to activate or otherwise initiate, augment and/or enhance the administration of the drug or therapeutic compound/active ingredient. For example, in use according to certain implementations, after the patch 10 has been applied to the skin 2, the augmentation layer can be configured to generate heat to facilitate the administration of the drug, as would be understood. It is understood that heat energy effectively enhances skin permeability of many drugs, which has been recently reviewed. Local heat exposure can alter any/all of the following: drug release from the formulation, stratum corneum barrier properties, and skin blood flow.

Further, local skin temperature of 42-43° C. can be maintained for up to 1-4 hours without damaging the skin. This approach is safe and effective for increasing drug flux (μg/cm² hr⁻¹), peak plasma drug concentrations, and area under the curve (AUC) in vivo. Exothermic reactions of iron oxidation can rapidly produce local heat upon air exposure and this strategy has been used to promote drug diffusion through the skin.

Accordingly, in certain examples, heat is generated via a chemical reaction. In certain examples, iron is provided as the heating agent, which can be activated via the removal of a cover 18 or protective liner 18 to expose the augmentation layer 16 to oxygen, as is shown in FIGS. 2A-2D. As the heat is generated, the microneedle layer 12 dissolves transdermally (shown at 12A), and the drug is released from the active layer 14 into the skin 2 of the subject. It is appreciated that in such examples, some drug such as NLX is delivered to the subject rapidly via the microneedles 1, while further administration occurs later as the drug leaves the active layer 14.

Importantly, in certain alternate implementations of the patch 10, the active layer 14 as separate from the microneedle layer 10 can be omitted. That is, certain implementations of the transdermal patch can comprise the microneedle layer 12 alone, or the microneedle layer 10 and the augmentation layer 16, with or without the liner 18, as would be readily appreciated. Accordingly, the present disclosure specifically contemplates an integration of the microneedle layer 12 and the active layer 14 or outright omission of an active layer 14 in certain patch 10 implementations.

Illustratively, it is understood that NLX is only active for ˜20-90 minutes after administration, and its effects may wear off long before the effects of the opioids. In cases where a patient has ingested large amounts of opioids, or highly potent opioids such as fentanyl, NLX may need to be re-administered every 2-3 minutes (these are known as “rescue” doses). The percent of patients requiring multiple NLX administrations to reverse overdose is increasing.

Rescue dosing can be a challenge if IV access is difficult to obtain/maintain, and there is a danger to using needles with patients regaining consciousness who may be combative. The commercially available intranasal NLX kit only contains two units, and after those doses are given the only available next step is to wait for first responders. On average, it takes 7 min after a 911 call for first responders to arrive on scene, but this time increases to >14 min in rural settings; 1 in 10 cases will wait for nearly half an hour for medical help to arrive. In opioid overdose situations, even a modest delay could threaten the life of the patient. This is a critical timeframe during which a dosage form is needed that can continuously deliver NLX until medical help arrives. The described patch 10 and associated methods are able to be administered quickly. Similarly, in alternate implementations utilizing therapeutic compounds for the treatment of certain other conditions, timing may be critical to treatment or relief of the presenting symptoms, and as such the augmentation layers can be configured to achieve the desired administration timeline.

Accordingly, in implementations like that of FIGS. 2A-2D, the microneedles 1 will begin immediate NLX delivery as they dissolve in the skin in ≤5 min (an achievable benchmark with dissolving microneedles). When exposed to air, such as via removal of the liner 18, the heating augmentation layer 18 increases local skin temperature and enhances the rate and extent of NLX delivery from the active layer 14 that will absorb through micropores created by the microneedles 1.

As described in the Examples herein, preliminary in vitro data demonstrate that microneedle pretreatment combined with increased skin temperature permits rapid NLX delivery (detection in 30 mins.), while also increasing NLX delivery 3-4× (vs. controls) by 1 hour. These combined data support the feasibility of this approach.

These in vitro studies were done to quantify NLX permeation through excised skin when microneedles and increased skin temperature were combined. Permeation studies are standard preclinical models for evaluating effects of formulation and treatments on skin drug absorption, and permeation data can be used to estimate in vivo plasma concentrations.

When microneedles 1 were used, NLX was detected in the receiver solution within 30 min and concentrations were 3-4× times higher vs controls by 1 hour when combined with heat. Naloxone permeation was increased through the entire study when microneedles and heat were combined, even after temperature was restored to 32° C.

These Examples show that with microneedle pretreatment and elevated skin temperature, the skin no longer presents a barrier or limitation to rapid diffusion/absorption of NLX or other drugs. Maximum NLX concentrations in the plasma for currently marketed dosage forms range from 1.2-10.3 ng/ml. Using NLX population clearance of 91 L/hr., a minimum target plasma concentration of 1.2 ng/ml, and flux calculated from preliminary data, a patch with area of ˜4.5 cm² (˜area of a US quarter) with these experimental conditions would be expected to achieve a minimum therapeutic plasma concentration of 1.2 ng/ml. This is described by the equation A=(CL*C)/J.

In vitro studies also show that NLX is detected at 5 min after application of the dissolving microneedles+topical NLX gel+heating element. With optimization of the proposed patch 10 components (dissolving microneedle layer 12, NLX gel/active layer 14, and heating component/augmentation layer 16) it is possible to deliver similar amounts of NLX, thus achieving suitable clinical benchmarks. Time of absorption will be significantly decreased with dissolving microneedles, allowing rapid NLX delivery necessary for reversing overdose.

In various implementations of the disclosed patch and methods of use, the administration time, that is, the time from the application of the patch to the skin of the subject until the detectability of the therapeutic compound in the serum of the subject will be less than about 30 minutes. In further implementations, the administration time will be less than about 20 minutes. In yet further implementations, the administration time will be less than about 15 minutes. In yet further implementations, the administration time will be less than about 10 minutes. In yet further implementations, the administration time will be less than about 5 minutes. In yet further implementations, the administration time will be less than about 3 minutes. In yet further implementations, the administration time will be less than about 2 minutes. In yet further implementations, the administration time will be less than about 1 minute.

In various implementations of the disclosed patch and methods of use, the administration time, that is, the time from the application of the patch to the skin of the subject until the detectability of the therapeutic compound in the serum of the subject will be more than about 30 minutes. In further implementations, the administration time will be more than about 60 minutes. In further implementations, the administration time will be more than about 90 minutes. In further implementations, the administration time will be more than about 120 minutes. Certain implementations make use of a liner to facilitate the administration time.

Further administration times are of course possible for differing therapeutics, and it is readily understood that because the administration is transdermal or topical, the administration can be continuous and the absorption/saturation can increase over time.

As used herein, the term “subject” refers to the target of administration, e.g., a human or an animal, such as a patient or a person in need of treatment. The term does not denote a particular age or sex. Thus, adult and newborn subjects, as well as fetuses, whether male or female, are intended to be covered, and includes those that have experienced or are suspected to have experienced, for example, an opioid overdose.

In further implementations, the term “subject” refers those that have experienced or are suspected to have experienced migraine, anaphylaxis, epilepsy, allergy, anxiety, asthma and COPD treatments, nausea, vomiting, cancer, diabetes, endocrine disorders, genetic disorders, hormonal disruptions, menopause, arrythmias, bacterial infections, viral infections, skin cancer, ocular disorders, depression, psychiatric disorders, HIV, AIDS, seizure disorders, and blood pressure changes.

### EXPERIMENTAL EXAMPLES

The following examples are put forth so as to provide those of ordinary skill in the art with a complete disclosure and description of how the articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### A. Example I

In one Example, a device comprising NLX-loaded microneedles 1 of the microneedle layer 12 were made of polyvinylpyrrolidone (PVP) or polymethyl vinyl ether/maleic anhydride (PMVE/MA) were fabricated with a 2-step process, using commercially available female microneedle templates, 600 or 800 μm length. Mechanical microneedle strength was tested using a rotational rheometer. Microneedle height was recorded before and after a compression test. Insertion depth of the microneedles was measured using optical coherence tomography to visualize the micropores. In vitro dissolution testing was performed to determine time for microneedles to completely dissolve. All data are in Table 1. The microneedles dissolved in as little as 2 min and penetrated the upper layers of the skin (all penetration depths >100 μm). In an initial permeation study, NLX was detected in receiver fluid at 5 min.

In another Example, proof-of-concept permeation studies were done to quantify NLX permeation through excised skin when microneedles and increased skin temperature were combined. Permeation studies are standard preclinical models for evaluating effects of formulation and treatments on skin drug absorption, and permeation data can be used to estimate in vivo plasma concentrations. Excised porcine skin was dermatomed to 1000 μm thickness (porcine skin is used because of its similarity to human skin and because many experiments can be performed with skin from one animal, reducing variability). Skin samples were treated with stainless steel arrays containing 50 microneedles, 800 μm long, which were discarded after use. Non-microneedle treated skin served as controls.

In this Example, skin samples were mounted into flow-through diffusion cells and allowed to equilibrate with the receiver solution (HEPES buffer, Ph 7.4, 37° C.). A 15% NLX-HCl gel in 2% hydroxyethylcellulose (HEC) was applied to the diffusion area. A thermoregulated arm which supports the diffusion cells was used to maintain skin temperature. Control samples were held at 32° C. (typical in vivo skin temperature). Heated skin samples were brought to 42° C. for 15 min and held there for 1 hr., after which the temperature was quickly brought down to 32° C. (rapidly achieved by adding chilled water to the circulating water bath that regulates temperature of the thermoregulated arm). Receiver solution was collected every 30 min for 6 hrs.

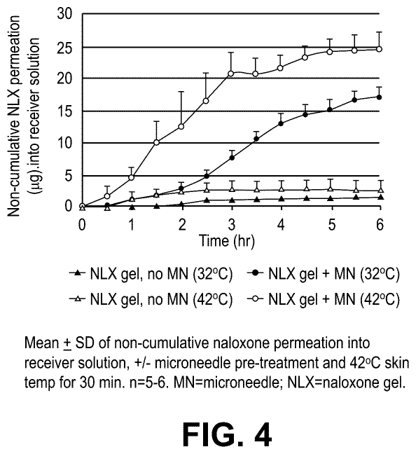

Results. When stainless steel microneedles were used, NLX was detected in the receiver solution within 30 min, and concentrations were 3-4× higher vs controls by 1 hr. when combined with 42° C. heat (FIG. 4). NLX permeation was increased through the entire study when microneedles and heat were combined, even after temperature was restored to 32° C. Using the equation A=(CI×Css)/Jss where CI is NLX population clearance of 91 L/hr, Css is a target plasma concentration of 1.2 ng/ml, and Jss is steady state flux of 24 g/cm² hr⁻¹ (calculated from the preliminary data cumulative permeation at 3-4 hrs.), a patch with area of −4.5 cm² (˜area of a US quarter) with the 15% NLX gel would achieve a therapeutic plasma concentration of 1.2 ng/ml. These preliminary studies utilized pretreatment with stainless steel microneedles, and the dissolving microneedles developed in this proposal would further reduce time for absorption to a clinically acceptable time of ≤5 min². With proper optimization of dissolving microneedles and NLX gel, time to peak delivery and steady state will both decrease, allowing rapid achievement of necessary plasma concentrations.

Further dissolving NLX microneedles that are currently being studied include 7.5% naloxone in 45%-50% PVP or 38.5%-40% PMVE/MA. These conditions are listed in Table 1, along with others. Further implementations are of course possible.

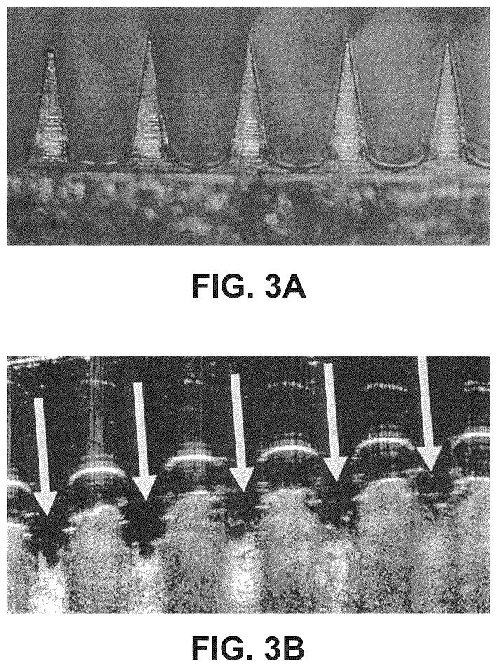

Heating augmentation layer design. Initial studies were conducted to optimize concentrations of excipients in the heating layer for achieving the desired increase in skin temperature (the heat is produced by an exothermic iron oxidation reaction). Studies were conducted using the following excipients and [c] ranges: 250-750 mg each of iron powder, charcoal, and vermiculite, 125-250 mg of NaCl, and 0.25-0.5 mL of water. Excised porcine skin was mounted in a 25 mm circular cap and warmed to normal skin temperature of 32° C. on the thermoregulated arm of a diffusion study apparatus (FIG. 3). A thermocouple thermometer probe was attached to the skin surface with Parafilm® to measure temperature. Combinations of heating layer excipients were prepared in tightly capped glass vials and exposed to air when poured onto the skin. Cotton gauze was used to cover the top of the open cap to allow proper aeration and mimic possible end product patch design. Skin temperature was measured every 15 sec for the first 15 min, then every 15 min for 3 hrs. Results. Fifteen preliminary studies were performed and when excipients were systematically varied one at a time, the maximum skin temperature ranged from 35.7° C. (at 79 min) to 59.6° C. (3.41 min). FIG. 4 shows the first 30 min for 2 trials that were close to the goal of rapidly achieving 42° C. For both trials the temperature returned to 32° C. by 45 min (data not shown). These data confirm that a rapid increase in skin temperature to ˜42° C. is achievable, and further optimization will focus on reaching/sustaining target temperature for 1 hr.

Initial studies according to another Example were conducted to optimize possible excipient concentrations in the heating augmentation layer 16 to achieve a desired increase in skin temperature (heat is produced by an exothermic iron oxidation reaction). Studies were conducted using the following: 250-750 mg each of iron powder, charcoal, and vermiculite, 125-250 mg of NaCl, and 0.25-0.5 mL of water. Fifteen preliminary studies were performed in which the maximum skin temperature ranged from 35.7° C. (at 79 min) to 59.6° C. (3.41 min).

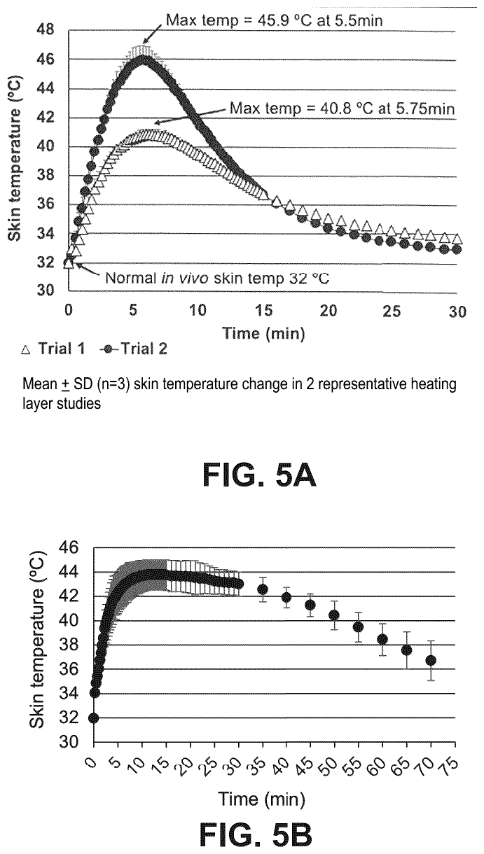

FIG. 5A shows the first 30 min for 2 trials that were close to the goal of rapidly achieving 42° C. Alternate implementations can be optimized so that the elevated temperature is maintained for a longer period of time, as would be appreciated.

The combined preliminary data of NLX permeation and skin heating studies demonstrate that all necessary resources and expertise are in place to conduct the proposed work, and findings support the feasibility of the envisioned patch design for treatment of opioid overdose.

Rigor and reproducibility. Scientific rigor will be maintained through all experiments. Diffusion studies of formulations will be carried out in replicates of 6-9. A power analysis was conducted by a biostatistician to detect statistically significant differences between groups. The statistician will be blinded to treatment groups during data analysis. Male and female animals will be included in equal numbers in the treatment groups, and data from each sex will be conducted separately and in combination to see if there are sex differences in the pharmacokinetic studies.

In another Example, parameters of microneedle application and temperature will be optimized to maximize NLX absorption through excised skin in vitro. The working hypothesis was that dissolving microneedles will permit detectable NLX absorption in ≤5 min compared to skin not treated with microneedles. It was further hypothesized that locally generated heat will increase the rate and extent of NLX absorption through the epidermal micropores, with a time of 15-20 min to achieve peak concentration. These targets were selected to align with pharmacokinetics of IM and intranasal delivery. Each patch layer has a benchmark to confirm successful performance.

Microneedles. Dissolving NLX microneedles fabricated with 98.5-100% polyvinylpyrrolidone (PVP) and 0.5-1.5% carboxymethylcellulose (CMC) were selected because they are biocompatible, produce microneedles of appropriate strength for skin insertion, and rapidly dissolve. They are made in a layer of 100 microneedles (600 μm length) using a silicone mold (Mpatch™ Microneedle Template). NLX solubility in each mixture will be determined and microneedles will be loaded with the highest NLX concentration. Compression and mechanical tests will be performed on a rotational rheometer with 25 mm disposable plate attachments, using methods modified from previous reports. After appropriate mechanical properties are achieved the microneedles will be attached to an adhesive backing and applied to excised porcine skin using finger pressure (to mimic real world conditions); the backing will ensure that the microneedles maintain skin contact. Optical coherence tomography will be used to measure insertion depth. Microneedles will be examined with SEM before and after 1, 2, and 5 min. insertion times.

Transdermal gel. Initial studies will be performed with a 15% NLX gel as described above. Stability studies will be performed at 32° C. and 42° C. to monitor for drug degradation (42° C. will only be applied for 1 hr. as the commercial patch would not be subjected to this temperature for long times). Samples will be collected at predetermined intervals; NLX will be quantified with HPLC. A drug-in-adhesive gel approach and other carriers like those discussed above will also be studied, which will simplify and optimize end patch design. For example, NLX solubility in various pressure-sensitive acrylic adhesives will be determined, and the highest concentration will be loaded.

Heating augmentation layer. Oxygen initiated exothermic reactions of iron oxidation will be used as the heating mechanism; this has been effective for other patches. A heating “pod” will be used, starting with the excipients and concentrations described in the preliminary data. The excipients will be contained in loosely woven gauze pouches and these heating pods will be kept in tightly capped vials to prevent air exposure.

Dermatomed porcine skin will be warmed to ˜32° C. to mimic in vivo skin temperature (described in preliminary data). The pod will be exposed to air and placed on the skin surface, and temperature will be measured at time points described above. Optimization of excipients, ratios, and gauze types will be performed in order to rapidly heat the skin to −42° C., maintained for 1 hour minimum, which is safe for human skin.

In vitro NLX permeation studies. NLX skin permeation will be quantified with static Franz diffusion cells, using similar conditions as described in the preliminary data. The dissolving NLX microneedles will first be inserted into the skin 2 and mounted into the diffusion cells. The NLX gel or drug-in-adhesive gel layer will be applied to the skin, covered by a layer of Parafilm®, followed by application of the heating pod (Parafilm® will prevent back absorption from the gels into the gauze). Skin temperature will be monitored with an infrared thermometer—the heating pod will be removed for measurements then quickly reapplied. Initial studies will be performed to measure skin temperature using the infrared thermometer on skin samples warmed to 32° C. and 42° C. with and without NLX gel on top. Differences in the measured skin temperature in the presence/absence of gel will allow a correction factor to be applied during data analysis. NLX in receiver samples will be analyzed via HPLC. Combinations of dissolving microneedles, NLX gel, and heating pods will be tested. Studies will be performed with n=6-9 replicates. Normal temperature (32° C.) and non-microneedle treated skin will serve as controls.

Data analysis. Non-cumulative and cumulative NLX concentrations in the receiver solution will be plotted as a function of time. Time of first detectable absorption, maximum point flux (μg/cm²/hr⁻¹), optionally, the time of maximum point flux (Tₘₐₓ), and area under the concentration-time curve (AUC) will be compared for all formulations and conditions (two-way ANOVA, p<0.05 considered significant). Maximum skin temperature and time to maximum skin temperature after application of heating pods are compared with paired t-tests.

It was hypothesized that dissolving microneedles quickly deliver NLX and achieve detectable drug in receiver fluid in ≤5 min. Further, that the augmentation or heating layer will quickly increase skin temperature and significantly enhance NLX permeation compared to unheated controls (with or without microneedles).

In another Example, in vivo proof of concept pharmacokinetic studies will be conducted. The working hypothesis was that the combined patch elements (dissolving microneedles, NLX gel, and heating layer) will synergistically increase NLX peak plasma concentration (Cₘₐₓ) and decrease time to achieve maximum concentration (Tₘₐₓ) in guinea pigs.

Animals. All animal procedures will be approved by the University of Iowa Institutional Animal Care and Use Committee. Hartley guinea pigs with indwelling cannulas for blood sampling will be used (Charles River Laboratories). Guinea pigs are a well-accepted small animal model commonly used in preclinical dermatology studies, as they are easy to handle and have sufficient blood volume for repeated sampling. A crossover study design will be used in 2 groups: 3 dosing arms, 4 animals/group. This approach allows us to use fewer animals, while also providing data on inter- and intra-animal variability. Animals will be dosed according to conditions in Table 2, with 24 hr. washouts between arms. The only difference between groups is that the heating pod will be applied in Group 2 but not Group 1.

A statistical power analysis (80% power, alpha=0.05) was performed based on variability in cumulative absorption at 1 hr. from the preliminary in vitro studies. A total of 8 animals will be used (n=4/group) for initial studies, and equal numbers of male and female animals will be included in each group. After initial studies are done and more information is available regarding variability, the power analysis will be adjusted if needed.

NLX treatments. All individual patch layers and combinations of layers that meet milestones (e.g., rapid dissolution in skin and administration time in ≤5 min, NLX delivery for minimum 2 hrs., Local skin temp of 42° C. for 1 hr.) will be tested in vivo. Baseline skin temperature will be recorded using an infrared thermometer. Gentle thumb pressure will be applied to insert the dissolving microneedles into the dorsal skin of the animal (an applicator will not be used so that the studies to mimic potential real-world situations). The NLX gel in the active layer (e.g., hydroxyethylcellulose (HEC) or drug-in-adhesive layer) will be applied over the same skin area, and then covered by the heating pod. For testing conditions without the heating pod, a fabricated rubber patch will protect the gel and help it stay in contact with the skin.

Pharmacokinetic studies. IV NLX-HCl (1 mg/kg) will be administered over 30 seconds through an indwelling cannula in the jugular vein; this dose was selected based on previous studies. IV dosing will provide the best estimate of systemic clearance and elimination half-life. For transdermal studies, hair on the dorsal surface will be removed with clippers 24 hrs. before the study (the hair will not be shaved, as this can compromise the skin barrier). In all studies, blood samples (200 Ml) will be collected into heparinized tubes every 5 min up to 30 min, and then every 30 min up to 2 hrs. Patches will be removed at 2 hrs., and samples will be drawn at 15 min intervals for another hr. to characterize elimination from the skin depot.

Data analysis. Drug will be extracted from the plasma and standard pharmacokinetic parameters will be determined including maximum plasma concentration (Cₘₐₓ), time of maximum plasma concentration (Tₘₐₓ), and area under the concentration-time curve from 0-2 hrs. (AUC_{0-2 hr}). Time of first detectable drug in the plasma will also be quantified. All endpoints will be compared with predictions from in vitro studies and will also be compared between arms in each animal, between animals in each group, and between groups. A mixed factorial ANOVA will be used to test for differences; p<0.05 will be considered significant. To address sex as a biological variable, data from male and female animals will first be analyzed separately to determine if sex affects any of the studied parameters. It is not expected that there will be significant differences because sex does not typically affect epidermal barrier properties or elimination.

### B. Example II

### Methods.

Heating Pod Design. Initial studies were conducted to optimize concentrations of excipients in the heating layer for achieving the desired increase in skin temperature (the heat is produced by an exothermic iron oxidation reaction). A total of 16 studies were conducted using the following excipients and concentration ranges: 250-1000 mg of iron powder, 150-750 mg each of charcoal and vermiculite; 62.5-250 mg of sodium chloride (NaCl), and 0.25-0.5 mL of water. Excised porcine skin was mounted in a 25 mm circular cap and warmed to approximate normal skin temperature of 32° C. on the thermoregulated arm of a diffusion study apparatus A thermocouple thermometer probe was attached to the skin surface with Parafilm® to measure the skin surface temperature. Combinations of heating layer excipients were prepared in small, sealed plastic pods with pre-fabricated pores that were covered with tape. The pods were placed on the surface of the skin samples and the tape was removed to expose the excipients to air. Skin temperature was measured every 15 sec for the first 15 min, then every 15 min for 3 hr. See, e.g., FIGS. 5A-B & 7.

Microneedle Fabrication and Mechanical Testing. Dissolving microneedle arrays (dMN) were fabricated. Each dMN array contained 100 MNs, 800 μm long, composed of 7.5% naloxone hydrochloride (NLX-HCL) in polyvinyl pyrrolidone (PVP, 55 KD molecular weight). The NLX-HCL/PVP solution was poured into female polydimethylsiloxane microneedle molds. After a 2 step process of centrifugation followed by pressure application, the dMNs were allowed to dry and were carefully extracted from the molds. The mechanical strength of fabricated dMN was assessed using an ARES G2 Rheometer (TA instruments, USA) in axial compression mode. The height of the needles was first measured using a stereomicroscope (SZ61, Olympus, Japan). To perform a compression test, the dMN arrays were placed on an aluminum plate with needles facing upward. The dMNs were compressed using a disc that lowered at a constant rate of 0.05 mm/sec until 5N axial force was reached, which was then held constant and applied for 30 sec. After removing the force, the dMN height was remeasured and % reduction in height was calculated. Optical coherence tomography (OCT) (Vivosight, Michelson Diagnostics Ltd. Kent, England) was utilized to measure ex vivo insertion depth and visualize dMN insertion into excised porcine skin. The skin was scanned before dMNs were applied, to obtain a baseline image of intact skin. The dMN arrays were inserted into the skin sample using gentle thumb pressure for 15 sec, and then the array was removed from the skin; OCT imaging was then repeated. The images were collected and analyzed using ImageJ software to measure depth of the pores created by the dMNs.

In Vitro Permeation Studies. Proof-of-concept studies were performed to quantify permeation of NLX-HCL (a hydrophilic model drug) through excised porcine skin when dMNs loaded with NLX-HCL, a topical NLX-HCL gel, and increased skin temperature (achieved via the heating pod) were combined. Permeation studies are standard preclinical models for evaluating effects of formulation and treatments on skin drug absorption. Excised porcine skin was dermatomed to 1000 μm thickness (porcine skin is used because of its similarity to human skin and because many experiments can be performed with skin from one animal, reducing variability).

One or two dMN arrays (for a total of 100 or 200 needles total) was applied to the porcine skin using gentle thumb pressure and the backing of the array was removed. The skin samples were then mounted into static glass Franz diffusion cells with 4.91 cm² diffusion area. A 15% NLX-HCL gel in 2% hydroxyethylcellulose (HEC) was applied topically over the skin, followed by the heating pod which was placed over the topical NLX-HCL gel, separated by a layer of parafilm. The heat reaction was initiated by removing the protective tape layer so the heating pod excipients could be exposed to air. The temperature of control samples was held at 32° C., which is approximate in vivo skin surface temperature. The receiver solution (the media into which drug is deposited after permeating the skin) was HEPES buffer, pH 7.4, maintained at physiological body temperature of 37° C.

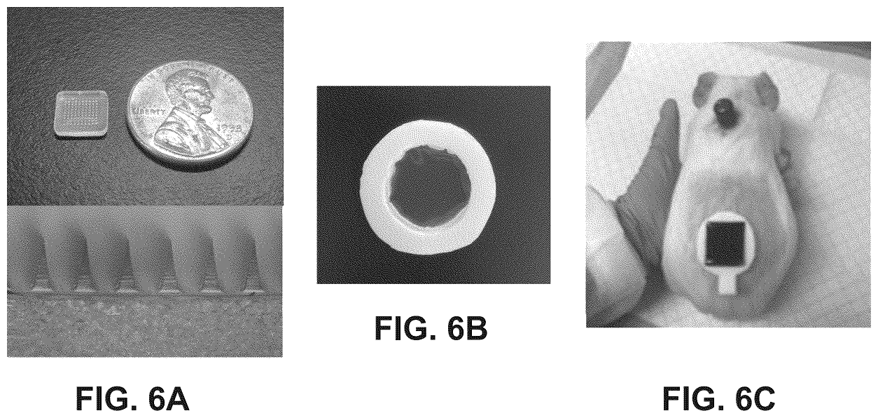

In Vivo Pharmacokinetics Studies. Preliminary in vivo data were collected in male (n=3) and female (n=2) albino Hartley guinea pigs (a common small animal model for dermal drug studies). All study procedures were IACUC approved. A crossover study design was used, dMN with and without use of heat (described in Table 5). The dorsal hair was clipped (not shaved) a day prior to studies. Either one or two dMN arrays (for a total of 100 or 200 dMNs total) were applied to the dorsal skin of the animal using gentle thumb pressure, and the backing of the dMN was removed. See FIGS. 6A-6C.

The 15% NLX-HCL gel was topically applied over the area where the dMNs had been inserted, and a heating pod was applied over the gel, separated by a layer of parafilm. The heat reaction was initiated by removing the protective tape cover. This was the same approach and conditions as used for the in vitro studies. Control conditions included no dMN and no heat (topical NLX gel only), and dMN+NLX-HCL gel (no heat). Blood samples were collected at 0, 5, 10, 15, 30, 60, 75, 90, and 120 min. Plasma was separated from the blood immediately after collection and stored at −80° C. until analysis. A thermocouple probe in contact with the skin during the in vivo studies was used to record the skin temperature at the same timepoints as blood sampling.

Following blood collection, NLX-HCL was extracted from the plasma using the following method: 100 μL of plasma was added to 500 μL ethyl acetate and 20 μL of internal standard (naltrexone hydrochloride) was added to achieve 50 ng/ml internal standard in the final reconstituted sample. The mixture was vortexed for 60 sec and centrifuged at 10,000 rpm for 15 min. The supernatant ethyl acetate layer was pipetted off and placed in a 5 mL glass tube under dry nitrogen to evaporate the organic layer. The dried residues were reconstituted with 100 μL 0.1% formic acid and analyzed using LC/MS.

Data Analysis. In vitro data: Non-cumulative and cumulative NLX-HCL concentrations that permeated into the receiver solution were plotted as a function of time. Time of first detectable NLX-HCL permeation and total permeation at 5, 15, 30, and 60 min was compared between the following conditions: 1) NLX-HCL gel applied to intact skin (no dMN); 2) 1 dMN+NLX-HCL gel; 3) 2 dMN+NLX-HCL gel. It is understood that when discussed herein, 1 dMN refers to a condition with a single, 100 microneedle array and 2 dMN refers to a condition with two arrays. All conditions were compared with and without the heating pod. In vivo data: NLX was extracted from the guinea pig plasma, quantified, and plotted vs. time. Time of first detectable drug in the plasma was quantified, and paired t-tests were applied to test for differences in plasma NLX-HCL concentrations between conditions at 5, 15, 30, and 60 min (all data were normalized to the weight of the animal at time of study). Total exposure to drug (area under the curve, AUC) was also compared between study conditions for the 0-5 min, 0-15 min, 0-30 min, and 0-60 min timeframes. For all analyses, p<0.05 was considered statistically significant.

### Results.

Heating Pod Studies. A total of 16 studies were performed, testing various combinations of excipients in the heating pods. In in vitro studies, the maximum recorded skin temperature ranged from 38.5° C. to 65.6° C., which occurred at times ranging from 2.3 min to 11.3 min. FIG. 5B depicts representative skin temperatures measured in vitro when the heating pod was used with excipients in concentrations listed in Table 3A. The decrease in temperature back to ˜32° C. occurred between 20 to 70 min. The heating pod containing iron, NaCl, charcoal, vermiculite, and water in the amounts seen in Table 3A was found to be most effective in raising and maintaining elevated skin temperature in a safe range (i.e., would not damage the skin) during in vitro studies. With these conditions, maximum skin temperature of 43.7° C. occurred at 11.3 min and temperature remained elevated (>32° C.) even at 70 min. It is of course fully appreciated that other formulations are of course possible and that alternate implementations utilize other combinations and amounts of excipients.

dMNs and Mechanical Testing. The height of 18 individual needles from 3 dMN arrays (6 needles from each array) were measured to determine % reduction in height after 30 sec of compression. There was a 22.2±3.5% reduction in height in the dMN arrays containing 7.5% NLX-HCL in 45% PVP (55 KD). The dMNs were mechanically strong and able to insert into excised porcine skin without breaking. OCT imaging confirmed that pores were created in the porcine skin treated with the dMNs, as shown for example in FIGS. 3A-3B. The average pore depth was 169.2±34.9 μm, which was calculated from the depth of 39 individual pores created from application of 3 different dMN arrays (13 pores created by each array).

In Vitro Studies. In the control condition when dMN were not used, NLX-HCL concentrations were below the limit of detection under both heated and non-heated conditions at all timepoints, demonstrating that the hydrophilic NLX-HCL compound does not permeate skin well. NLX-HCL was detected in receiver solution at 5 min when dMNs were used, regardless of whether or not the heating pod was applied. When considering the 1 dMN condition, NLX-HCL concentrations in receiver solution ranged from 2-9× higher in the heated vs non-heated conditions; for the 2 dMN studies the NLX-HCL concentrations were 1.4-2.6× higher when heated conditions were used (with the exception of the 5 min timepoint, at which the concentrations were approximately equal between heat vs. no heat). For both the 1 and 2 dMN conditions, the heated studies had significantly higher NLX-HCL concentrations in receiver solution vs. non-heated conditions at 15 and 30 min (p<0.05). The 2 dMN condition produced significantly higher concentrations in receiver solution compared to the 1 dMN at 15, 30, and 60 min for non-heated conditions (p<0.05). There was no significant difference in permeation from 1 vs 2 dMN under heated conditions at any timepoint except at 5 min, when the 2 dMN condition produced higher concentrations than the 1 dMN condition (p<0.05). See Table 3B.

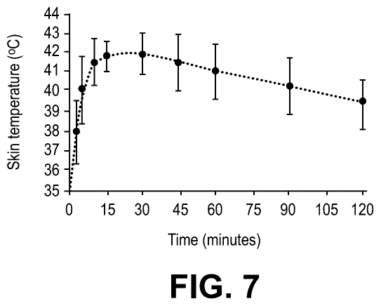

In Vivo Studies. The mean±SD baseline temperature of the guinea pig skin surface during pharmacokinetics studies with heating pods was 35.9±0.4° C. (n=5 animals in 19 total studies). When the heating pod was placed on the patch design and exposed to air, skin temperature began increasing immediately, achieving a maximum of 42.1±0.9° C. at 30 min. The temperature remained elevated throughout the study, and decreased to 39.8±1.3° C. at 120 min (this was still 3.9° C. higher than baseline), as shown in FIG. 7.

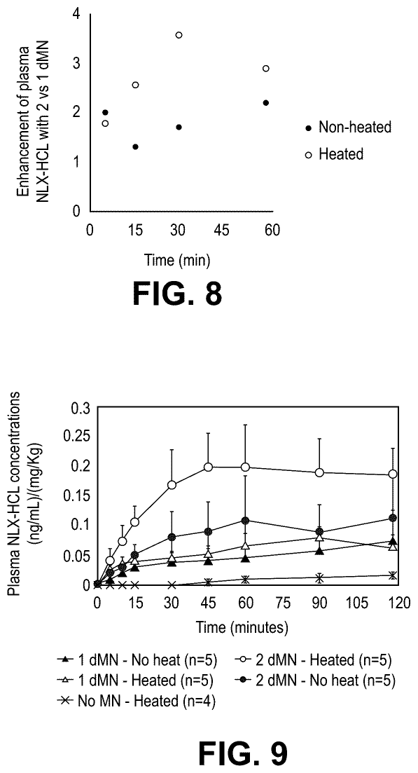

In all studies with dMN (regardless of whether a heating pod was used), NLX-HCL was detected in the plasma at 5 min (see FIG. 9, Table 5). Under non-heated conditions the 2 dMN condition resulted in concentrations that were 2.1, 1.6, 2.0, and 2.3× higher than the 1 dMN condition at 5, 15, 30, and 60 min., respectively (see FIG. 8). Under heated conditions the enhancement ratio between 2 vs 1 dMN was 1.8, 2.6, 3.6, and 2.9 at 5, 15, 30, and 60 min respectively (see FIG. 8).

As shown in FIG. 9, applied heat in the 2 dMN condition enhanced plasma concentrations of NLX-HCL by 2.1, 2.2, 2.1, and 1.8× at 5, 15, 30, and 60 min, respectively. For the 1 dMN condition, the enhancement in plasma concentration achieved with heat (vs no heat) was 2.4, 1.3, 1.2, and 1.4 at 5, 15, 30, and 60 min, respectively. When comparing plasma concentrations (normalized by dose delivered and kg of animal body weight) at 5, 15, 30, and 60 min there were no statistically significant differences at any time point between 1 vs 2 dMN (heated or not heated), and plasma concentrations under heated conditions were not significantly higher than those obtained using dMN without heat for 2 or 1 dMN arrays. However, when area under the normalized concentration curve (AUC, (ng*min/ml)/(mg/kg)) was calculated and compared for the 0-5, 0-15, 0-30, and 0-60 min ranges, the application of heat resulted in significantly higher AUC for all time ranges for 2 dMN when compared to the same conditions without heat (p<0.05). Additionally, under heated conditions the AUC was significantly higher for 2 dMN vs 1 dMN at all time ranges tested (p<0.05).

These are important findings because AUC is a measure of total exposure to a drug over a given timeframe. Thus, these findings demonstrate that the use of heat (via a heating pod) significantly enhances dermal absorption and exposure for a hydrophilic drug delivered with use of dMNs.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Although the disclosure has been described with reference to preferred embodiments, persons skilled in the art will recognize that changes may be made in form and detail without departing from the spirit and scope of the disclosed apparatus, systems, and methods.

1-47. (canceled)

48. A transdermal patch for administration of naloxone to a subject in need thereof, comprising:
a) a dissolving microneedle layer;
b) an active layer; and
c) an augmentation layer.

49. The transdermal patch of claim 48, wherein the augmentation layer is configured to generate heat when exposed to oxygen.

50. The transdermal patch of claim 48, wherein the augmentation layer contains one or more of iron, perlite, fine saw dust, fine coir, sodium acetate, graphene oxide and/or nanostructures.

51. The transdermal patch of claim 49, further comprising a protective liner configured to protect the augmentation layer from exposure to oxygen.

52. The transdermal patch of claim 51, wherein removal of the protective liner exposes the augmentation layer to oxygen and the augmentation layer subsequently generates heat.

53. The transdermal patch of claim 48, wherein naloxone is contained in the active layer.

54. The transdermal patch of claim 48, wherein naloxone is contained in the dissolving microneedle layer.

55. The transdermal patch of claim 48, wherein naloxone is contained in both the active layer and the dissolving microneedle layer.

56. A transdermal patch for the administration of naloxone to a subject, comprising:
a) a microneedle layer comprising microneedles comprising naloxone and a binder;
b) an active layer comprising naloxone and a carrier; and
c) an augmentation layer configured to generate local heat on skin of the subject.

57. The transdermal patch of claim 56, wherein the binder is polyvinylpyrrolidone.

58. The transdermal patch of claim 56, wherein the binder is polymethyl vinyl ether/maleic anhydride.

59. The transdermal patch of claim 56, wherein the microneedles are dissolving microneedles.

60. The transdermal patch of claim 56, wherein the microneedles are non-dissolving microneedles.

61. The transdermal patch of claim 56, wherein the microneedle layer comprises both dissolving and non-dissolving microneedles.

62. A method for administering a therapeutic compound to a subject, comprising:
applying a transdermal patch to the skin of a subject, wherein the transdermal patch comprises:
a) a microneedle layer comprising microneedles comprising the therapeutic compound; and
b) an augmentation layer configured to generate local heat on skin of the subject continuing the application of the transdermal patch for an administration time.

63. The method of claim 62, wherein the transdermal patch further comprises a protective liner configured to protect the augmentation layer from oxygen.

64. The method of claim 63, further comprising removing the protective liner to expose the augmentation layer to oxygen, wherein the exposure to oxygen causes the augmentation layer to generate heat.

65. The method of claim 62, wherein the administration time is less than about 30 minutes.

66. The method of claim 62, wherein the administration time is less than about 15 minutes.

67. The method of claim 62, wherein the administration time is more than about 30 minutes.
